(19) United States
(12) Patent Application Publication
(10) Pub. No.: US 2026/0162798 A1
(43) Pub. Date: Jun. 11, 2026

(54) INFORMATION PROCESSING DEVICE, INFORMATION PROCESSING METHOD, AND COMPUTER READABLE MEDIUM

(71) Applicant: Mitsubishi Electric Corporation, Tokyo (JP)

(72) Inventor: Yoshiyuki NORIMATSU, Tokyo (JP)

(73) Assignee: Mitsubishi Electric Corporation, Tokyo (JP)

(21) Appl. No.: 19/538,446

(22) Filed: Feb. 12, 2026

Related U.S. Application Data
(63) Continuation of application No. PCT/JP2023/036251, filed on Oct. 4, 2023.

(51) Int. Cl. G16H 20/10; G06N 3/0455; G06N 3/08

(52) U.S. Cl. CPC G16H 20/10; G06N 3/0455; G06N 3/08

(57) ABSTRACT

A data acquisition unit (110) acquires, as training data concerning a plurality of treatments, time-series data including: a variant feature that varies according to treatment; an invariant feature that does not vary according to treatment; and a categorical variable concerning the treatment. An encoder learning unit (120) optimizes an encoder that predicts a treatment result of time t+1 that is 1 step ahead of any given time t, using the training data. A decoder learning unit (130) optimizes a decoder that predicts a treatment result of each time following time t+1, using the training data. An estimation unit (140) estimates a treatment result of each of a plurality of times following time t+1, concerning the plurality of treatments, using the optimized encoder and the optimized decoder.

### CROSS REFERENCE TO RELATED APPLICATION

This application is a Continuation of PCT International Application No. PCT/JP2023/036251, filed on Oct. 4, 2023, which is hereby expressly incorporated by reference into the present application.

### TECHNICAL FIELD

The present disclosure relates to information processing for estimating treatment results concerning a plurality of treatments conducted at a plurality of time points.

### BACKGROUND ART

Various techniques are known that estimate counterfactual treatment results taking into account the treatment type and a treatment dosage.

For example, Non-Patent Literature 1 discloses a method of estimating counterfactual treatment results taking into account a treatment and a treatment dosage at a single time point using a generative adversarial network (GAN).

### CITATION LIST

### Non-Patent Literature

Non-Patent Literature 1: Bica, I., Jordon, J., and van der Schaar, M.: Estimating the effects of continuous-valued interventions using generative adversarial networks, Proceedings of the 33rd International Conference on Neural Information Processing Systems (NeurIPS), pp. 16434-16445 (2020)
Non-Patent Literature 2: Zaheer, M., Kottur, S., Ravanbakhsh, S., Poczos, B., Salakhutdinov, R. R., and Smola, A. J. Deep sets. In Advances in Neural Information Processing Systems (NeurIPS), pp. 3391-3401 (2017)

### SUMMARY OF INVENTION

### Technical Problem

The method of Non-Patent Literature 1 estimates the treatment result taking into account a treatment and a treatment dosage at a single time point. However, this method is unable to estimate the treatment result when a plurality of treatments are performed at intervals with different types of treatments and different treatment dosages.

An objective of the present disclosure is to enable estimation of treatment results concerning a plurality of treatment conducted at a plurality of time points.

### Solution to Problem

An information processing device of the present disclosure includes:
a data acquisition unit to acquire, as training data concerning a plurality of treatments, time-series data including: a variant feature that varies according to treatment; an invariant feature that does not vary according to treatment; and a categorical variable concerning treatment;
an encoder learning unit to optimize an encoder that predicts a treatment result of time t+1 that is 1 step ahead of any given time t, using the training data;
a decoder learning unit to optimize a decoder that predicts a treatment result of each time following time t+1, using the training data; and
an estimation unit to estimate a treatment result of each of a plurality of times following time t+1, concerning the plurality of treatments, using the optimized encoder and the optimized decoder.

### Advantageous Effects of Invention

According to the present disclosure, it is possible to estimate treatment results concerning a plurality of treatments conducted at a plurality of time points.

### BRIEF DESCRIPTION OF DRAWINGS

FIG. 1 is a graph showing examples of a plurality of treatments in Embodiment 1.

FIG. 2 is a graph showing examples of a dose-response curve in Embodiment 1.

FIG. 3 is a graph showing examples of data patterns of treatment results in Embodiment 1.

FIG. 4 is a configuration diagram of an information processing device 100 in Embodiment 1.

FIG. 5 is a configuration diagram of a data acquisition unit 110 in Embodiment 1.

FIG. 6 is a configuration diagram of an encoder learning unit 120 in Embodiment 1.

FIG. 7 is a configuration diagram of a decoder learning unit 130 in Embodiment 1.

FIG. 8 is a flowchart of an information processing method in Embodiment 1.

FIG. 9 is a flowchart of step S10 in Embodiment 1.

FIG. 10 is a diagram showing an overview of a model of an encoder in Embodiment 1.

FIG. 11 is a diagram showing an overview of the model of the encoder in Embodiment 1.

FIG. 12 is a diagram showing an overview of the model of the encoder in Embodiment 1.

FIG. 13 is a flowchart of step S20 in Embodiment 1.

FIG. 14 is a flowchart of step S20 in Embodiment 1.

FIG. 15 is a diagram showing an overview of a generator G^{en} in Embodiment 1.

FIG. 16 is a diagram showing an overview of a model of a treatment dosage discriminator D_{d} in Embodiment 1.

FIG. 17 is a diagram showing an overview of a model of a treatment discriminator D_{w} in Embodiment 1.

FIG. 18 is a graph showing an overview of treatment dosage discrimination in Embodiment 1.

FIG. 19 is a graph showing an overview of treatment discrimination in Embodiment 1.

FIG. 20 is a diagram showing an overview of a model of a decoder in Embodiment 1.

FIG. 21 is a diagram showing an overview of a model of the decoder in Embodiment 1.

FIG. 22 is a flowchart of step S30 in Embodiment 1.

FIG. 23 is a flowchart of step S30 in Embodiment 1.

FIG. 24 is a diagram showing an overview of a generator G^{de} in Embodiment 1.

FIG. 25 is a flowchart of step S40 in Embodiment 1.

FIG. 26 is a hardware configuration diagram of the information processing device 100 in Embodiment 1.

### DESCRIPTION OF EMBODIMENTS

In the embodiment and drawings, the same elements or equivalent elements are denoted by the same reference signs. Description of elements denoted by the same reference signs as described elements may be appropriately omitted or simplified.

Arrows in the drawings mainly represent flows of data or flows of process.

### Embodiment 1

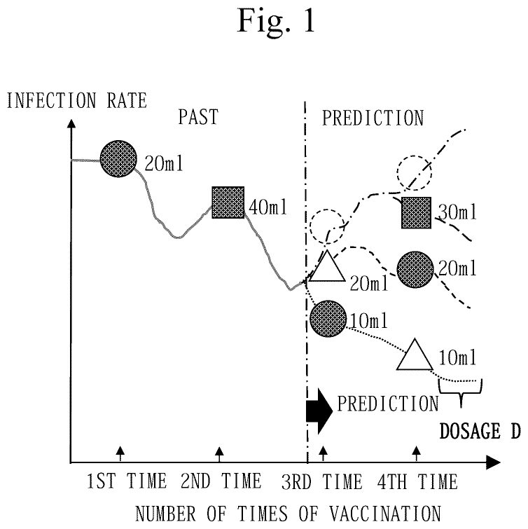

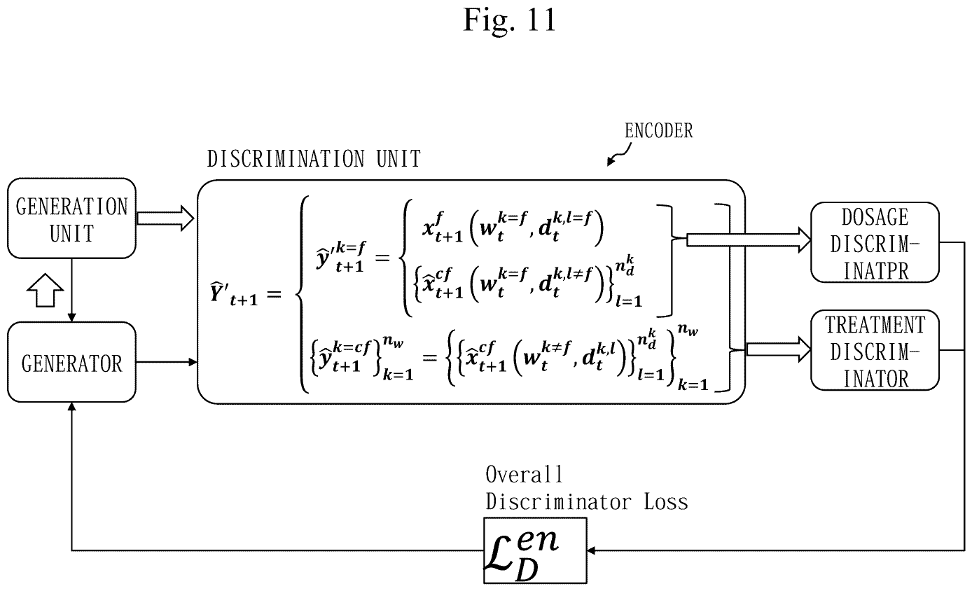

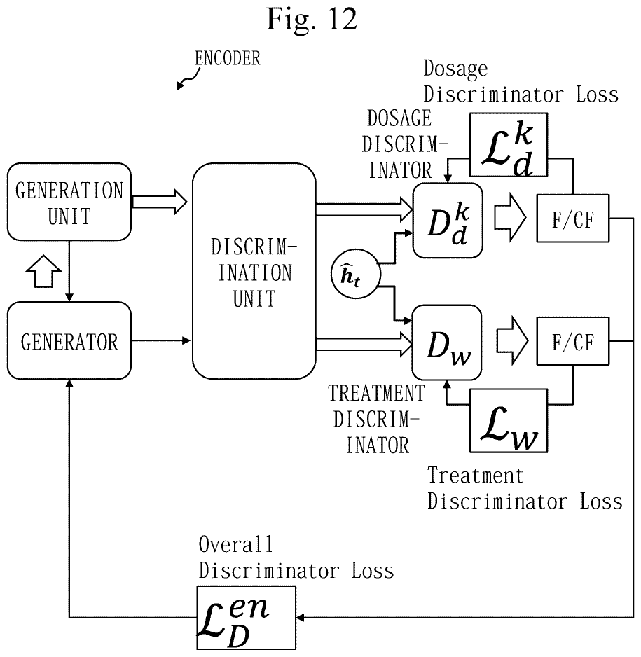

Embodiment 1 will be described with referring to FIG. 1 through FIG. 26.

### Explanation on Overview

A plurality of treatments imply conducting different types of treatments with different treatment dosages at intervals. A treatment dosage refers to an amount of an item used in treatment. Treatment can also be referred to as processing.

FIG. 1 illustrates examples of a plurality of treatments. FIG. 1 represents conducting treatments a plurality of times using a plurality of types of vaccines with various dosages of treatments. A dotted circle, a circle, a square, and a triangle represent the types of treatment w, and an amount attached to each figure represents a treatment dosage d. A dotted circle represents no vaccinations performed. A solid circle represents treatment with a vaccine A. A square represents treatment with a vaccine B. A triangle represents treatment with a vaccine C.

A relationship between the treatment dosage and the treatment result has different characteristics according to the type of treatment, and is expressed by a Dose-Response Curve.

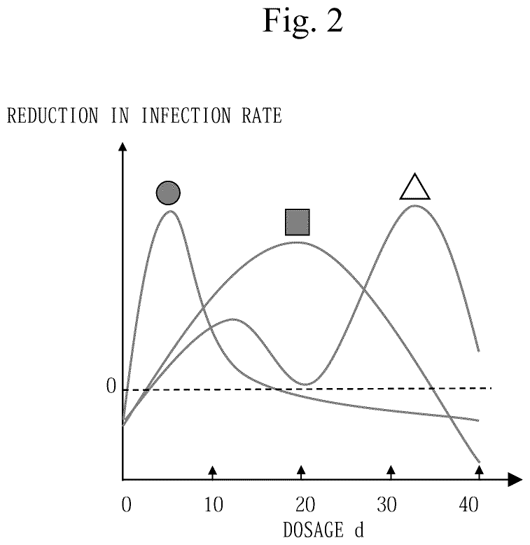

FIG. 2 shows examples of a dose-response curve. In FIG. 2, the Dose-Response Curve represents a relationship between a vaccine dose and a reduction in infection rate. The relationship between the vaccine dose and the reduction in infection rate has different characteristics according to the vaccine type.

Factual data refers to data of observed treatment results.

Counterfactual data refers to data of unobserved treatment results.

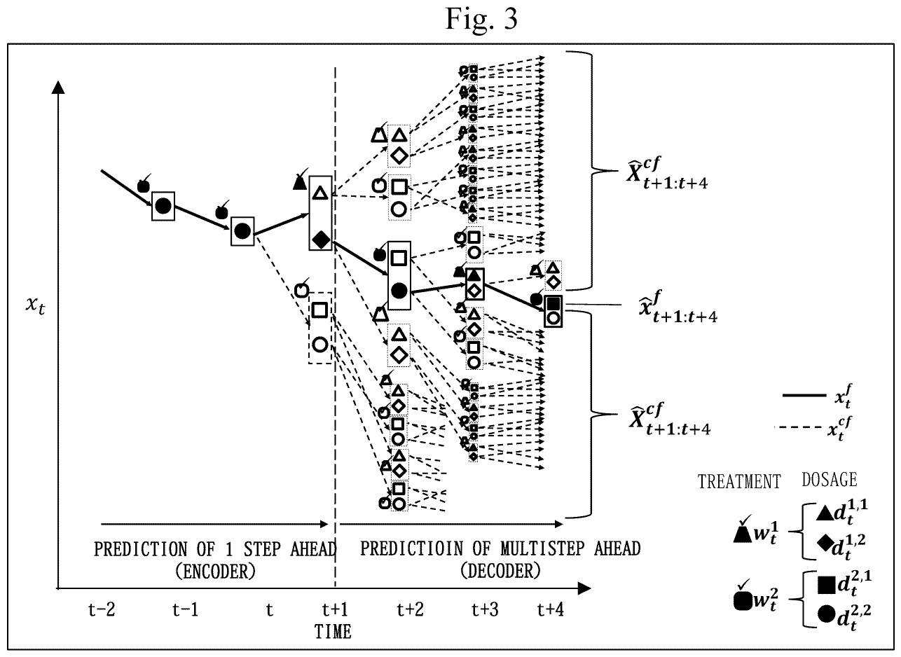

FIG. 3 shows examples of data patterns of treatment results. In FIG. 3, there are two types of treatments {wₜ¹, wₜ²}, and each treatment has two types of treatment dosages {dₜ^{1,1}, dₜ^{1,2}} and {dₜ^{2,1}, dₜ^{2,2}}.

In FIG. 3, when a treatment is performed four times after time t, 256 types of treatment results X_{t+1:t+4} can be obtained. In this case, actually observed factual data x^{f} consists of one pattern, and the remaining 255 patterns are counterfactual data X\hat{ }^{cf}.

In order to know the most effective treatment pattern, it is necessary to know the treatment results of all patterns, including counterfactual data. The counterfactual data can be predicted using a time-series prediction model such as an LSTM

However, when counterfactual data is predicted using a time-series prediction model such as an LSTM, the prediction model tends to overfit the observed factual data, resulting in poor prediction accuracy for the counterfactual data.

Note that LSTM stands for Long Short Term Memory.

In the technology described in Non-Patent Literature 1, in order to prevent overfitting, a generator is trained so that factual data and counterfactual data which is generated by the generator using GAN cannot be distinguished by a discriminator.

However, the technology described in Non-Patent Literature 1 can only estimate the treatment result at a single time point. In other words, treatment results at a plurality of time points cannot be estimated.

Note that GAN stands for Generative Adversarial Network.

Therefore, in Embodiment 1, treatment results at a plurality of time points are estimated using a time-series GAN suited to the time-series data.

### Description of Configuration

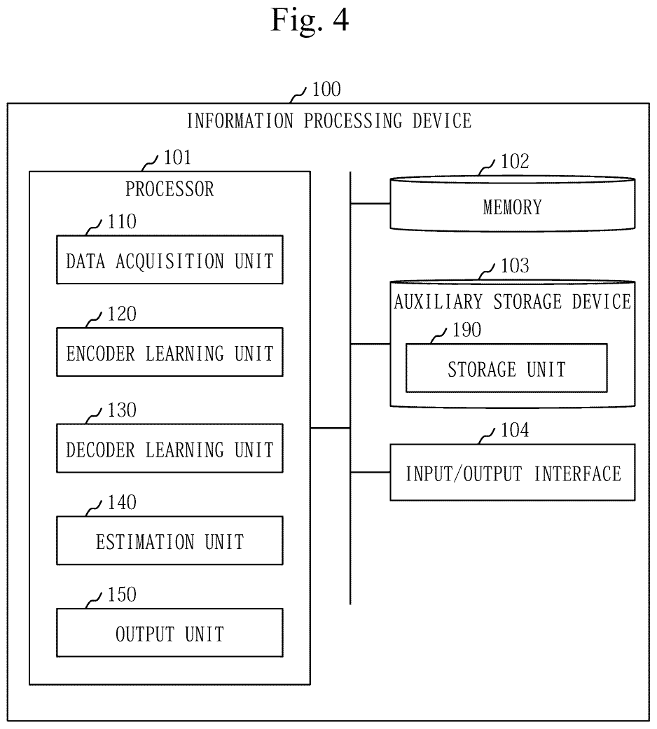

With referring to FIG. 4, a configuration of an information processing device 100 will be described.

The information processing device 100 is a computer equipped with hardware devices such as a processor 101, a memory 102, an auxiliary storage device 103, and an input/output interface 104. These hardware devices are connected to each other via a signal line.

The processor 101 is an IC that performs computational processing and controls the other hardware devices. For example, the processor 101 is a CPU.

Note that IC stands for Integrated Circuit.

Note that CPU stands for Central Processing Unit.

The memory 102 is a volatile or non-volatile storage device. The memory 102 is also called a main storage device or main memory. For example, the memory 102 is a RAM. Data stored in the memory 102 is saved in the auxiliary storage device 103 as needed.

Note that RAM stands for Random Access Memory.

The auxiliary storage device 103 is a non-volatile storage device. For example, the auxiliary storage device 103 is a ROM, an HDD, or a flash memory; or a combination of these. Data stored in the auxiliary storage device 103 is loaded onto the memory 102 as needed.

Note that ROM stands for Read Only Memory.

Note that HDD stands for Hard Disk Drive.

The input/output interface 104 is a port where an input device and an output device are connected. For instance, the input/output interface 104 is a USB terminal, the input device consists of a keyboard and a mouse, and the output device is a display. A communication device is an example of the input and output devices. Input to and output from the information processing device 100 are performed via the input/output interface 104.

Note that USB stands for Universal Serial Bus.

The information processing device 100 comprises elements such as a data acquisition unit 110, an encoder learning unit 120, a decoder learning unit 130, an estimation unit 140, and an output unit 150. These elements are implemented by software.

The auxiliary storage device 103 stores an information processing program necessary for causing the computer to function as the data acquisition unit 110, the encoder learning unit 120, the decoder learning unit 130, the estimation unit 140, and the output unit 150. The information processing program is loaded into the memory 102 and is executed by the processor 101.

Furthermore, the auxiliary storage device 103 stores an OS. At least part of the OS is loaded into the memory 102 and is executed by the processor 101.

The processor 101 executes the information processing program while also executing the OS.

Note that OS stands for Operating System.

Input and output data of the information processing program are stored in a storage unit 190.

The auxiliary storage device 103 functions as the storage unit 190. However, a storage device such as the memory 102, a register within the processor 101, and a cache memory within the processor 101 may function as the storage unit 190, either in place of the memory 102 or in conjunction with the memory 102.

The information processing program can be computer-readably recorded (stored) in a non-volatile recording medium such as an optical disc and a flash memory.

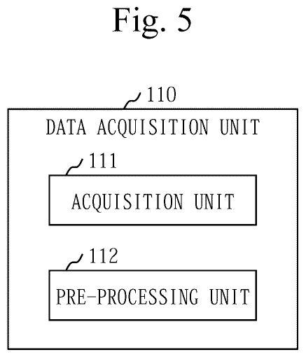

FIG. 5 shows a configuration of the data acquisition unit 110.

The data acquisition unit 110 includes elements such as an acquisition unit 111 and a pre-processing unit 112.

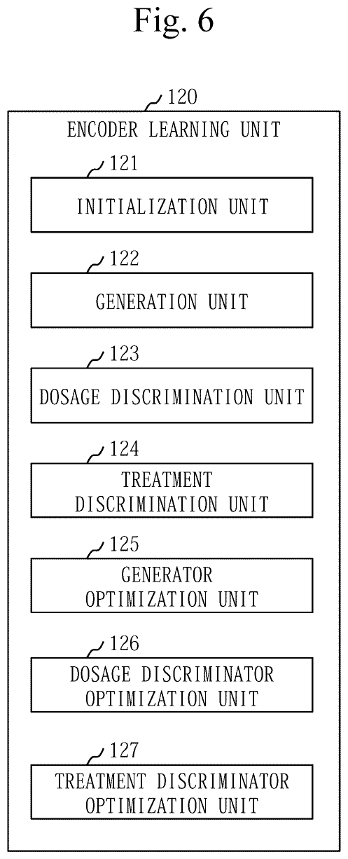

FIG. 6 shows a configuration of the encoder learning unit 120.

The encoder learning unit 120 includes elements such as an initialization unit 121, a generation unit 122, a treatment dosage discrimination unit 123, a treatment discrimination unit 124, a generator optimization unit 125, a treatment dosage discriminator optimization unit 126, and a treatment discriminator optimization unit 127.

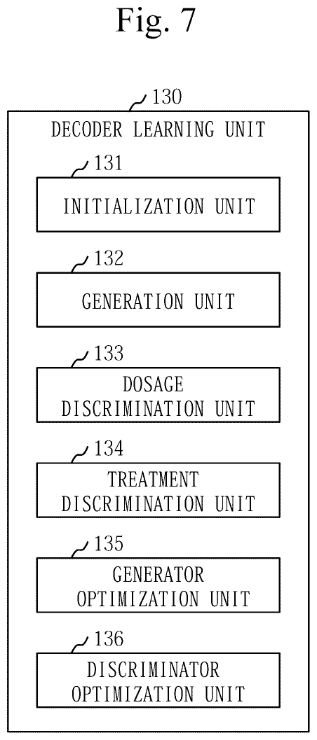

FIG. 7 shows a configuration of the decoder learning unit 130.

The decoder learning unit 130 includes elements such as an initialization unit 131, a generation unit 132, a treatment dosage discrimination unit 133, a treatment discrimination unit 134, a generator optimization unit 135, and a discriminator optimization unit 136.

### Description of Operation

An operation procedure of the information processing device 100 corresponds to an information processing method. The operation procedure of the information processing device 100 also corresponds to a processing procedure conducted by the information processing program.

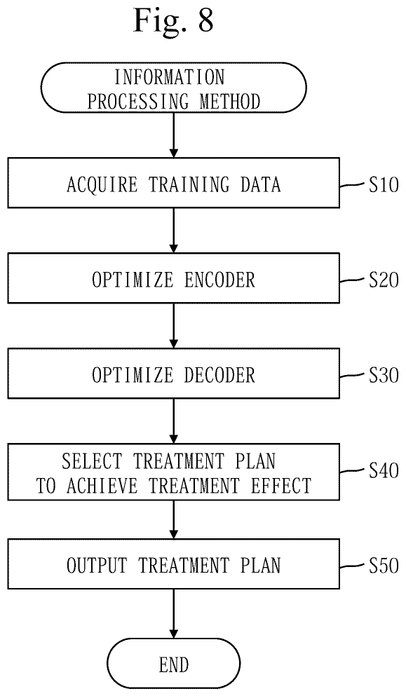

With referring to FIG. 8, the information processing method will be described.

In step S10, the data acquisition unit 110 acquires training data.

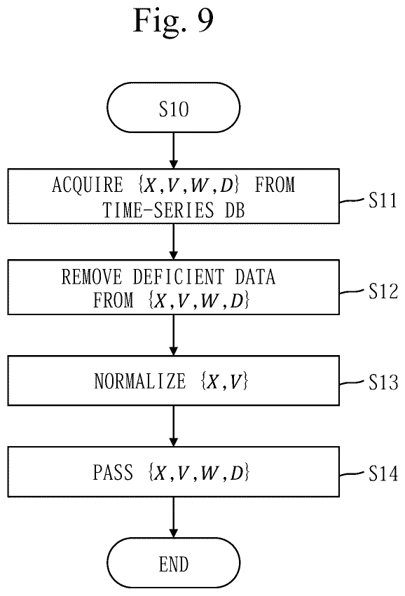

With referring to FIG. 9, a procedure of step S10 will be described.

In step S11, the acquisition unit 111 acquires {X, V, W, D} from a time-series database and passes {X, V, W, D} to the pre-processing unit 112.

Note that {X, V, W, D} is training data used for learning.

The time-series database is a database where time-series data and static data are registered. For example, the storage unit 190 functions as the time-series database.

Note that “X” represents a variant feature. A variant feature is a time-varying covariate that varies according to treatment.

Note that “V” represents an invariant feature. An invariant feature is a baseline covariate that does not vary according to treatment.

Note that “W” represents a categorical variable concerning treatment.

Note that “D” represents a categorical variable concerning treatment dosage.

The categorical variable W is expressed as follows using a number k for treatment and a total number nₖ of treatment types.

$$W ∋ {w}_{t}^{k} = \{ {w}_{t}^{1} = 1 , {w}_{t}^{2} = 2 , … , {w}_{t}^{{n}_{k}} = {n}_{k} \} \qquad [ Formula 101 ]$$

The categorical variable D is expressed as follows. The categorical variable D for a case where k=2 is exemplified.

$$\begin{matrix}D ∋ {d}_{t}^{k , l} \\ k = 2 , {d}_{t}^{2 , l} = \{ {d}_{t}^{2 , l} = 10 , {d}_{t}^{2 , 2} = 20 , … , {w}_{t}^{2 , {n}_{k}} = 50 \}\end{matrix} \qquad [ Formula 102 ]$$

Note that {X, V, W, D} is observed for each individual i, and is expressed as a set of time-series data and static data, as indicated in Expression (1).

$$\begin{matrix}[ Formula 103 ] & \\ \begin{matrix}\{ X , V , W , D \} = {\{ {\{ {x}_{t}^{f , ( i )} , {w}_{t}^{k = f , ( i )} , {d}_{t}^{k = f , l = f , ( i )} \}}_{t = 1}^{{t}_{max}^{( i )}} , {v}^{( i )} \}}_{i = 1}^{N} \\ N : 1 , {t}_{max}^{( i )} : 2 , {w}_{t}^{k = f , ( i )} : 3 , {d}_{t}^{k = f , l = f , ( i )} : 4\end{matrix} & ( 1 )\end{matrix}$$

Note that:
[symbol] 1 represents a number of individuals observed;
[symbol] 2 represents a time duration of the time-series data;
[symbol] 3 indicates a type of treatment applied at time t where “k=f” means that the treatment was actually observed; and
[symbol] 4 represents a treatment dosage applied at time t where “l=f” means that the treatment dosage was actually observed.

In step S12, the pre-processing unit 112 removes, from {X, V, W, D}, data of an individual i in which the value of at least one of X, V, W, D is missing.

The data of the individual i is expressed as follows.

$$\{ {\{ {x}_{t}^{f , ( i )} , {w}_{t}^{k = f , ( i )} , {d}_{t}^{k = f , l = f , ( i )} \}}_{t = 1}^{{t}_{max}^{( i )}} , {v}^{( i )} \qquad [ Formula 104 ]$$

In step S13, the pre-processing unit 112 normalizes each of X and V.

For instance, X and V are normalized such that their average becomes 0 and their variance becomes 1.

In step S14, the pre-processing unit 112 passes {X, V, W, D} to the encoder learning unit 120.

Returning to FIG. 8, the explanation resumes from step S20.

In step S20, the encoder learning unit 120 optimizes an encoder.

The encoder predicts a treatment result of time t+1. Time t+1 is time that is 1 step ahead of any given time t.

Specifically, the encoder learning unit 120 optimizes parameters of the encoder.

In other words, the encoder learning unit 120 finds the optimal parameter values for the encoder.

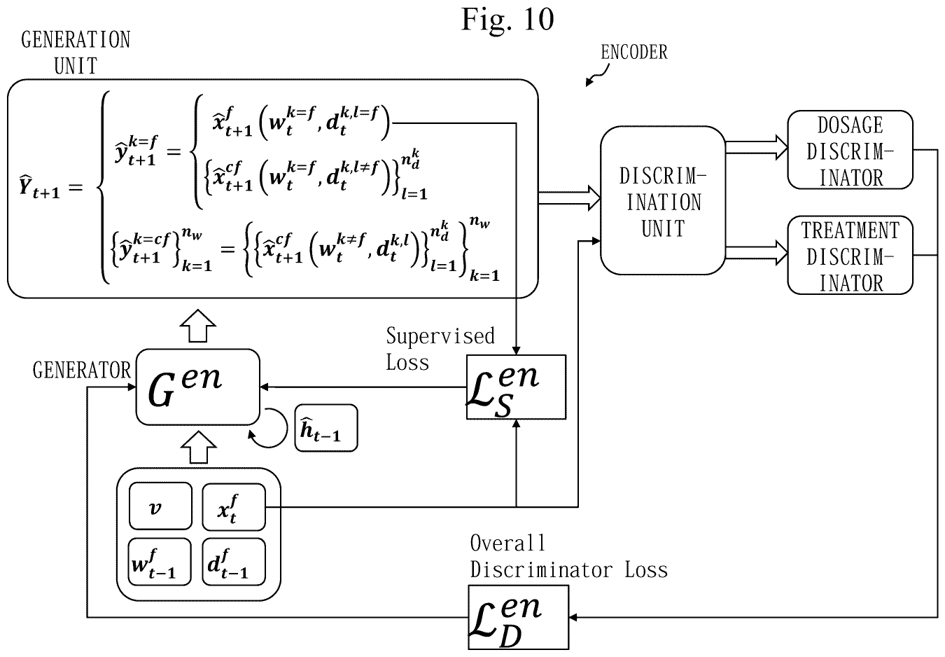

FIGS. 10 to 12 show overviews of a model of the encoder.

The encoder has a generator G^{en}. The encoder further has a treatment dosage discriminator D_{d} and a treatment discriminator D_{w} for each treatment type.

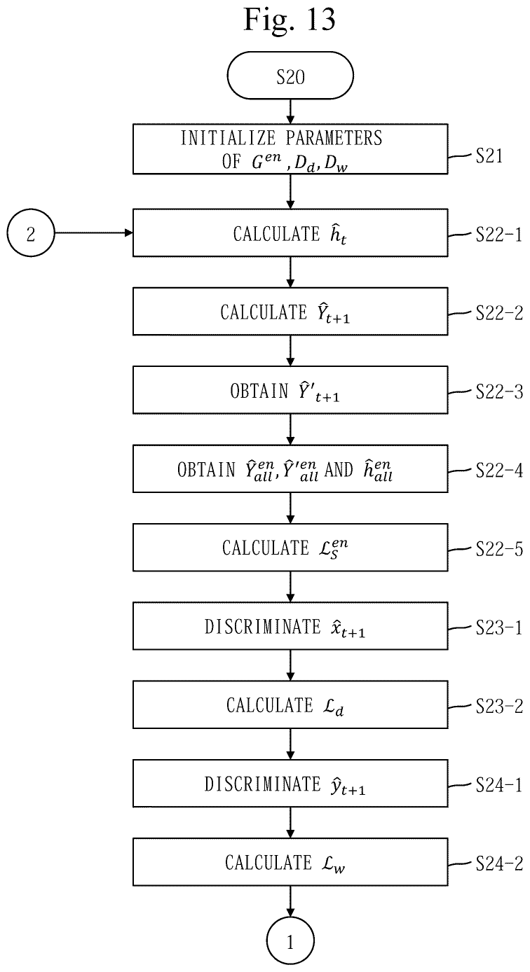

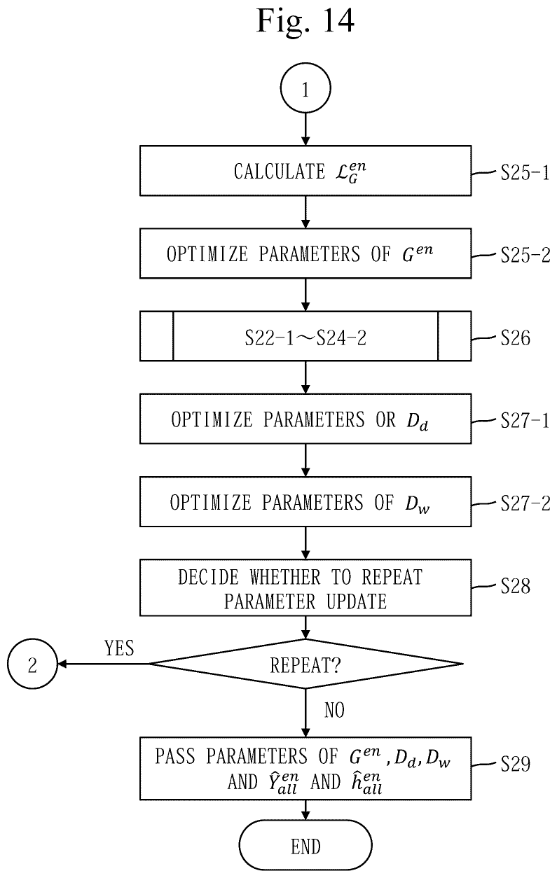

With referring to FIG. 13 and FIG. 14, a procedure of step S20 will be described.

In step S21, the initialization unit 121 initializes the parameters of each of the generator G^{en}, the treatment dosage discriminator D_{d}, and the treatment discriminator D_{w}.

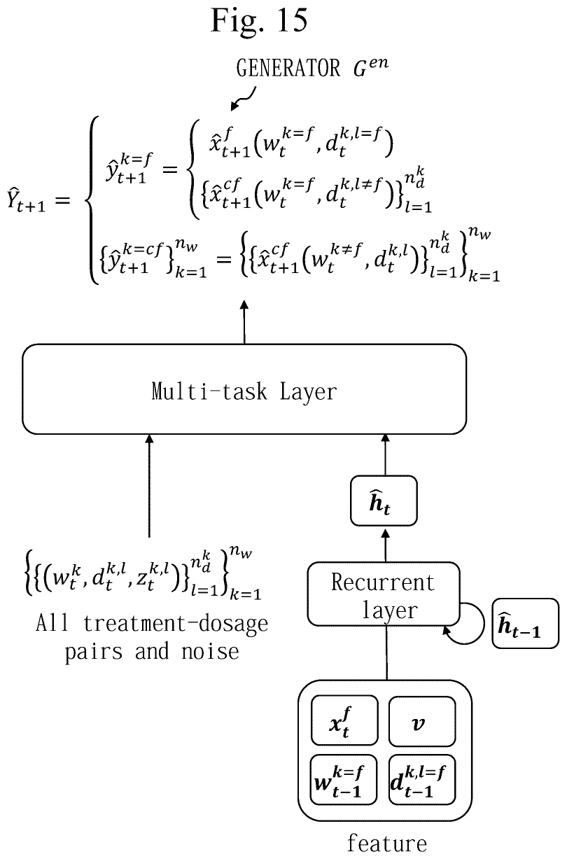

FIG. 15 illustrates an overview of the generator G^{en}.

In the generator G^{en}, parameters of both a recurrent layer and a multi-task layer are initialized.

The recurrent layer could be a well-known deep neural network. Examples of the well-known deep neural network include an RNN, an LSTM, a GRU, and a bidirectional LSTM. Note that RNN stands for recurrent neural network, LSTM for long short term memory, and GRU for gated recurrent unit.

The multi-task layer represents a neural network with a plurality of outputs.

The treatment dosage discriminator D_{d} is expressed as follows.

$${D}_{d} = {\{ {D}_{d}^{k} \}}_{k = 1}^{{n}_{k}} \qquad [ Formula 201 ]$$

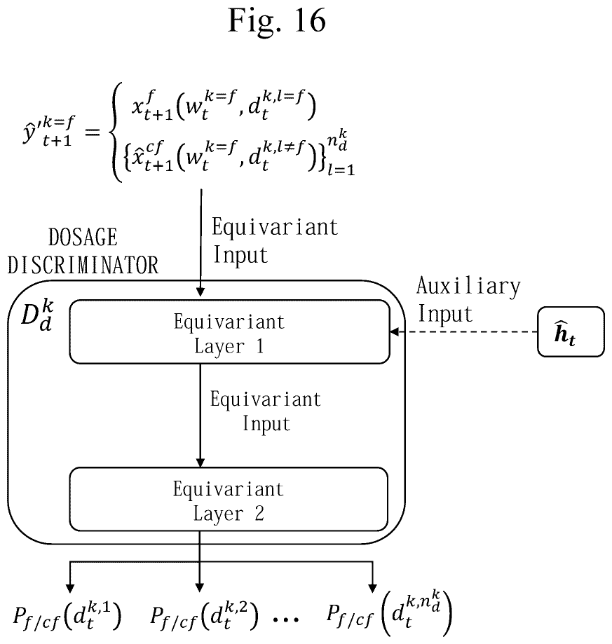

FIG. 16 shows an overview of a model of the treatment dosage discriminator D_{d}.

In the treatment dosage discriminator D_{d}, parameters of an equivariant layer 1 of D_{d}^{k} and equivariant layer 2 of D_{d}^{k} are initialized.

A model proposed in Non-Patent Literature 2 can be used as the equivariant layer.

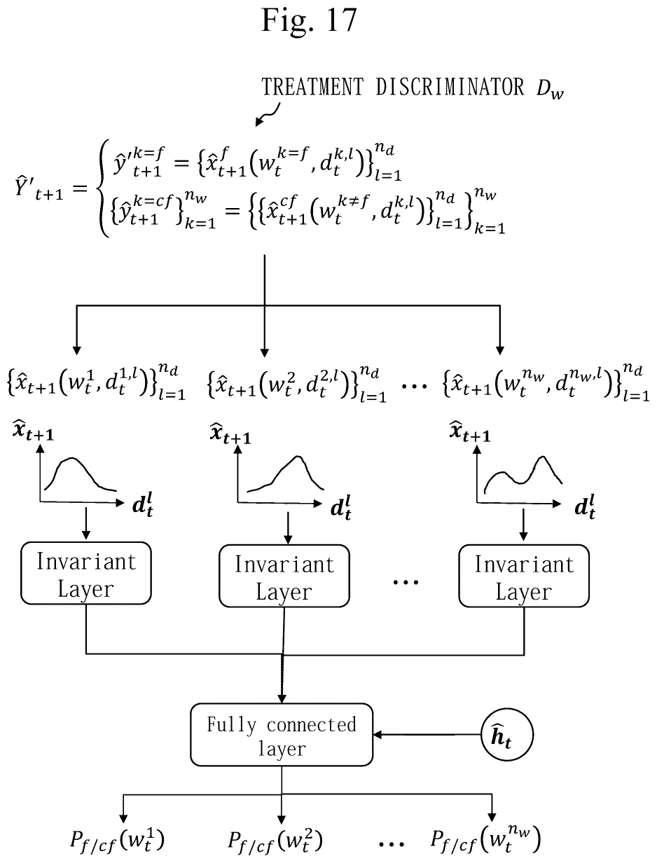

FIG. 17 shows an overview of a model of the treatment discriminator D_{w}.

In the treatment discriminator D_{w}, the parameters of each invariant layer and the parameters of a fully connected layer are initialized.

A model proposed in Non-Patent Literature 2 can be used for the invariant layer.

The fully connected layer represents a neural network of the fully connected layer.

An example of initialization is Xavier initialization or He initialization.

Returning to FIG. 13, the explanation resumes from step S22-1.

In step S22-1, the generation unit 122 calculates an intermediate state h\hat{ }ₜ of the recurrent layer of the generator G^{en}.

The intermediate state h\hat{ }ₜ is calculated by inputting elements extracted from {X, V, W, D} and an intermediate state h\hat{ }ₜ₋₁ of the recurrent layer of the generator G^{en} to the recurrent layer of the generator G^{en} recursively.

The elements extracted from {X, V, W, D} are expressed as follows.

$$\{ {x}_{t}^{f} , v , {w}_{t - 1}^{k = f} , {d}_{t - 1}^{k , l = f} \} \qquad [ Formula 202 ]$$

In step S22-2, the generation unit 122 calculates a set Y\hat{ }ₜ₊₁.

The set Y\hat{ }ₜ₊₁ is calculated by inputting the intermediate state h\hat{ }ₜ and a combination of treatment, treatment dosage, and noise to the multi-task layer of the generator G^{en}.

The combination of treatment, treatment dosage, and noise is expressed as follows.

$${\{ {\{ ( {w}_{t}^{k} , {d}_{t}^{k , l} , {z}_{t}^{k , l} ) \}}_{l = 1}^{{n}_{d}^{k}} \}}_{k = 1}^{{n}_{w}} \qquad [ Formula 203 ]$$

The set Y\hat{ }ₜ₊₁ is a set of fact and counterfact at time t+1.

The set Y\hat{ }+1 is expressed by Expression (10) where “k=f” signifies a fact and “k=cf” signifies a counterfact.

$$\begin{matrix}[ Formula 204 ] & \\ {\hat{Y}}_{t + 1} = \{ \begin{matrix}{\hat{y}}_{t + 1}^{k = f} = \{ \begin{matrix}{\hat{x}}_{t + 1}^{f} ( {w}_{t}^{k = f} , {d}_{t}^{k , l = f} ) \\ {\{ {\hat{x}}_{t + 1}^{cf} ( {w}_{t}^{k = f} , {d}_{t}^{k , l \neq f} ) \}}_{l = 1}^{{n}_{d}^{k}}\end{matrix} \\ {\{ {\hat{y}}_{t + 1}^{k = cf} \}}_{k = 1}^{{n}_{w}} = {\{ {\{ {\hat{x}}_{t + 1}^{cf} ( {w}_{t}^{k \neq f} , {d}_{t}^{k , l} ) \}}_{l = 1}^{{n}_{d}^{k}} \}}_{k = 1}^{{n}_{w}}\end{matrix} & ( 10 )\end{matrix}$$

In step S22-3, the generation unit 122 obtains a set Y\hat{ }′ₜ₊₁.

The set Y\hat{ }′ₜ₊₁ is obtained by replacing a factual element x\hat{ }^{f} with an observed value x^{f} in Expression (10).

The set Y\hat{ }′ₜ₊₁ is expressed by Expression (11).

$$\begin{matrix}[ Formula 205 ] & \\ {\hat{Y}}_{t + 1}^{′} = \{ \begin{matrix}{\hat{y}}_{t + 1}^{′ k = f} = \{ \begin{matrix}{x}_{t + 1}^{f} ( {w}_{t}^{k = f} , {d}_{t}^{k , l = f} ) \\ {\{ {\hat{x}}_{t + 1}^{cf} ( {w}_{t}^{k = f} , {d}_{t}^{k , l \neq f} ) \}}_{l = 1}^{{n}_{d}^{k}}\end{matrix} \\ {\{ {\hat{y}}_{t + 1}^{k = cf} \}}_{k = 1}^{{n}_{w}} = {\{ {\{ {\hat{x}}_{t + 1}^{cf} ( {w}_{t}^{k \neq f} , {d}_{t}^{k , l} ) \}}_{l = 1}^{{n}_{d}^{k}} \}}_{k = 1}^{{n}_{w}}\end{matrix} & ( 11 )\end{matrix}$$

In step S22-4, the generation unit 122 obtains a set Y\hat{ }^{en}, a set Y\hat{ }′^{en}, and a set h\hat{ }^{en} for each individual i.

The set Y\hat{ }^{en} is obtained by calculating Expression (12).

The set \hat{ }′^{en} is obtained by calculating Expression (13).

The set h\hat{ }^{en} is obtained by calculating Expression (14) with the recurrent layer of the generator G^{en}.

Note that “all” signifies all the individuals i.

$$\begin{matrix}[ Formula 206 ] & \\ {\hat{Y}}_{all}^{en} = {\{ {\{ {\hat{Y}}_{t + 1}^{( i )} \}}_{t = 1}^{{t}_{max}^{( i )} - 1} \}}_{i = 1}^{N} & ( 12 )\end{matrix}$$

$${{\hat{Y}}^{′}}_{all}^{en} = {\{ {\{ {{\hat{Y}}^{′}}_{t + 1}^{( i )} \}}_{t = 1}^{{t}_{max}^{( i )} - 1} \}}_{i = 1}^{N} \qquad ( 13 )$$

$${\hat{h}}_{all}^{en} = {\{ {\{ {\hat{h}}_{t + 1}^{f , ( i )} \}}_{t = 1}^{{t}_{max}^{( i )} - 1} \}}_{i = 1}^{N} \qquad ( 14 )$$

In step S22-5, the generation unit 122 calculates a loss function L_{S}^{en}.

The loss function L_{S}^{en} calculates a mean squared error (MSE) of the factual element x\hat{ }^{f} in Expression (10) and the observed value x^{f}.

The loss function L_{S}^{en} is expressed by Expression (15).

$$\begin{matrix}[ Formula 207 ] & \\ {ℒ}_{S}^{en} = {\sum }_{i = 1}^{N} {\sum }_{t = 1}^{{t}_{max}^{( i )} - 1} {( {x}_{t + 1}^{f , ( i )} ( {w}_{t}^{k = f} , {d}_{t}^{k , l = f} ) - {\hat{x}}_{t + 1}^{f , ( i )} ( {w}_{t}^{k = f} , {d}_{t}^{k , l = f} ) )}^{2} & ( 15 )\end{matrix}$$

In step S23-1, the treatment dosage discrimination unit 123 discriminates each of elements xₜ₊₁ (x^{f}, x\hat{ }^{cf}) that constitute the set Y\hat{ }′ₜ₊₁ in Expression (11). Expression (11) is calculated in each time step.

Each element xₜ₊₁ is discriminated by “1” or “0” where “1” signifies a fact (f) and “0” signifies a counterfact (cf).

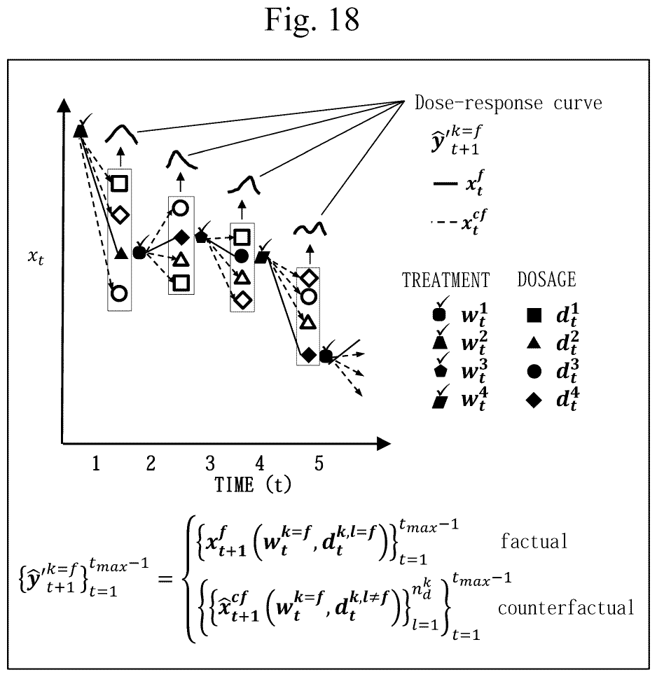

FIG. 18 shows an overview of treatment dosage discrimination.

Each element xₜ₊₁ is discriminated as follows.

First, the treatment dosage discrimination unit 123 extracts an element y\hat{ }′ₜ₊₁ (k=f) from the set Y\hat{ }′^{en} in Expression (13).

The element y\hat{ }′ₜ₊₁ to be extracted is expressed by Expression (20).

$$\begin{matrix}[ Formula 208 ] & \\ {{\hat{y}}^{′}}_{t + 1}^{k = f} = \{ \begin{matrix}{x}_{t + 1}^{f} ( {w}_{t}^{k = f} , {d}_{t}^{k , l = f} ) \\ {\{ {\hat{x}}_{t + 1}^{cf} ( {w}_{t}^{k = f} , {d}_{t}^{k , l \neq f} ) \}}_{l = 1}^{{n}_{d}^{k}}\end{matrix} & ( 20 )\end{matrix}$$

Also, the treatment dosage discrimination unit 123 extracts the element h\hat{ }ₜ from the set h\hat{ }^{en} in Expression (14).

Next, the treatment dosage discrimination unit 123 selects the treatment dosage discriminator D_{d} (k=f) of the observed treatment from the treatment dosage discriminator D_{d}.

The treatment dosage discriminator D_{d} is expressed as follows.

$${D}_{d} = {\{ {D}_{d}^{k} \}}_{k = 1}^{{n}_{k}} \qquad [ Formula 209 ]$$

Then, the treatment dosage discrimination unit 123 inputs the element y\hat{ }′ₜ₊₁ and the element h\hat{ }ₜ to the treatment dosage discriminator D_{d} (k=f) to discriminate each element xₜ₊₁.

Each element xₜ₊₁ is discriminated according to the following procedure.

First, the treatment dosage discrimination unit 123 inputs the element y\hat{ }′ₜ₊₁ to the equivariant layer 1 as an equivariant input.

Also, the treatment dosage discrimination unit 123 inputs the element h\hat{ }ₜ to the equivariant layer 1 as an auxiliary input.

Then, the treatment dosage discrimination unit 123 inputs an output from the equivariant layer 1 to the equivariant layer 2 as an equivariant input.

As a result, the equivariant layer 2 outputs a discrimination result of each element xₜ₊₁.

In step S23-2, the treatment dosage discrimination unit 123 calculates a total sum L_{d}.

The total sum L_{d} is calculated as follows.

First, for each individual i, the treatment dosage discrimination unit 123 discriminates each element x\hat{ }ₜ₊₁.

The element x\hat{ }ₜ₊₁ to be discriminated is expressed as follows.

$${\{ {\{ {\{ {\hat{x}}_{t + 1}^{( i )} ( {w}_{t}^{k = f} , {d}_{t}^{k , l} ) \}}_{l = 1}^{{n}_{d}^{k}} \}}_{t = 1}^{{t}_{max}^{( i )} - 1} \}}_{i = 1}^{N} \qquad [ Formula 210 ]$$

Next, for each treatment k, the treatment dosage discrimination unit 123 calculates a loss function L_{d}^{k} to determine a loss.

The loss function L_{d}^{k} is expressed by Expression (21).

$$\begin{matrix}[ Formula 211 ] & \\ {ℒ}_{d}^{k} = {\sum }_{i = 1}^{N} {\sum }_{t = 1}^{{t}_{max}^{( i )} - 1} [ log {D}_{d}^{k} ( {x}_{t + 1}^{f , ( i )} ( {w}_{t}^{k = f} , {d}_{t}^{k , l = f} ) ) + {\sum }_{l = 1}^{{n}_{d}^{k}} log ( 1 - {D}_{d}^{k} ( {\hat{x}}_{t + 1}^{cf , ( i )} ( {w}_{t}^{k = f} , {d}_{t}^{k , l \neq f} ) ) ) ] & ( 21 )\end{matrix}$$

Then, the treatment dosage discrimination unit 123 calculates the total sum L_{d} of the loss.

The total sum L_{d} is expressed by Expression (22).

$$\begin{matrix}[ Formula 212 ] & \\ {ℒ}_{d} = {\sum }_{k = 1}^{{n}_{w}} {ℒ}_{d}^{k} & ( 22 )\end{matrix}$$

In step S24-1, the treatment discrimination unit 124 discriminates each of elements y\hat{ }ₜ₊₁ that constitute the set Y\hat{ }′ₜ₊₁ in Expression (11). Note that Expression (11) is calculated in each time step.

Each element y\hat{ }ₜ₊₁ is discriminated by “1” or “0” where “1” signifies a fact (f) and “0” signifies a counterfact (cf).

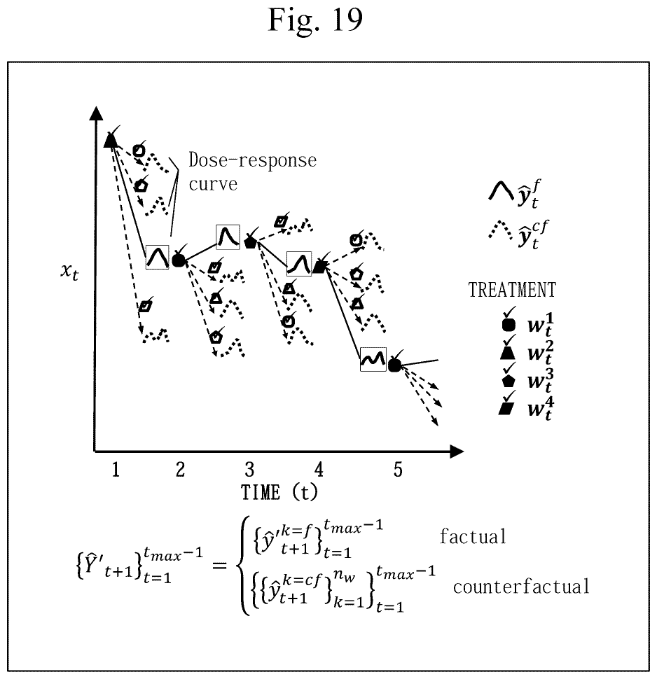

FIG. 19 shows an overview of the treatment discrimination.

Each element y\hat{ }ₜ₊₁ is discriminated in the following way.

First, the treatment discrimination unit 124 extracts the element y\hat{ }′ₜ₊₁ of Expression (11) from the set Y\hat{ }′^{en} in Expression (13).

Also, the treatment discrimination unit 124 extracts the element h\hat{ }ₜ from the set h\hat{ }^{en} in Expression (14).

Then, the treatment discrimination unit 124 inputs the element y\hat{ }′ₜ₊₁ and the element h\hat{ }ₜ to the treatment discriminator D_{w} to discriminate each element y\hat{ }ₜ₊₁.

Each element y\hat{ }ₜ₊₁ is discriminated by the following procedure.

First, the treatment discrimination unit 124 inputs each of the following elements of the element y\hat{ }′ₜ₊₁ to the invariant layers.

$${{\hat{y}}^{′}}_{t + 1}^{k = f} \qquad [ Formula 213 ]$$

$${\{ {\hat{y}}_{t + 1}^{k = cf} \}}_{k = 1}^{{n}_{w}}$$

Then, the treatment discrimination unit 124 inputs outputs of the invariant layers and the element h\hat{ }ₜ to the fully connected Layer.

As a result, the fully connected layer outputs a discrimination result for each element y\hat{ }ₜ₊₁.

In step S24-2, for each individual i, the treatment discrimination unit 124 discriminates each element y\hat{ }ₜ₊₁.

The element y\hat{ }ₜ₊₁ to be discriminated is expressed as follows.

$${\{ {\{ {\{ {\hat{y}}_{t + 1}^{k , ( i )} \}}_{k = 1}^{{n}_{k}} \}}_{t = 1}^{{t}_{max}^{( i )} - 1} \}}_{i = 1}^{N} \qquad [ Formula 214 ]$$

Then, the treatment discrimination unit 124 calculates a loss function L_{W}.

The loss function L_{W} is expressed by Expression (31).

$$\begin{matrix}[ Formula 215 ] & \\ {ℒ}_{w} = {\sum }_{i = 1}^{N} {\sum }_{t = 1}^{{t}_{max}^{( i )} - 1} [ log {D}_{w} ( {{\hat{y}}^{′}}_{t + 1}^{k = f , ( i )} ) + {\sum }_{k = 1}^{{n}_{w}} log ( 1 - {D}_{w} ( {\hat{y}}_{t + 1}^{k = cf , ( i )} ) ) ] & ( 31 )\end{matrix}$$

In step S25-1, the generator optimization unit 125 calculates a loss function L_{G}^{en} of the generator G^{en} using a loss (L_{S}^{en}), the total sum L_{d}, and a loss (L_{W}).

The loss (L_{S}^{en}) is a value obtained by calculating Expression (15).

The total sum L_{d} is a value obtained by calculating Expression (22).

The loss (L_{W}) is a value obtained by calculating Expression (31).

The loss function L_{G}^{en} is expressed by Expression (40) where “ad” is a hyperparameter indicating a degree of consideration taken for the total sum L_{d}, and “aw” is a hyperparameter indicating a degree of consideration taken for the loss (L_{W}).

$$\begin{matrix}[ Formula 216 ] & \\ {ℒ}_{G}^{en} = {ℒ}_{S}^{en} - {α}_{d} {ℒ}_{d} - {α}_{w} {ℒ}_{w} & ( 40 )\end{matrix}$$

In step S25-2, the generator optimization unit 125 optimizes the parameters of the generator G^{en}. As a result, the parameters of the generator G^{en} are updated.

The parameters of the generator G^{en} are optimized such that an output value of the loss function L_{G}^{en} becomes minimum. An optimization technique such as known stochastic gradient descent is used for the optimization.

In step S26, the encoder learning unit 120 uses the optimized parameters of the generator G^{en} to execute the processes of step S22-1 to step S24-2.

In step S27-1, the treatment dosage discriminator optimization unit 126 optimizes the parameters of the treatment dosage discriminator D_{d}. As a result, the parameters of the treatment dosage discriminator D_{d} are updated.

The treatment dosage discriminator D_{d} is expressed as follows.

$${D}_{d} = {\{ {D}_{d}^{k} \}}_{k = 1}^{{n}_{k}} \qquad [ Formula 217 ]$$

Specifically, the treatment dosage discriminator optimization unit 126 optimizes the parameter of each element D_{d}^{k} of the treatment dosage discriminator D_{d}.

The parameter of each element D_{d}^{k} is optimized such that the loss (L_{d}^{k}) becomes minimum. An optimization method such as the known stochastic gradient descent method is used for the optimization.

The loss (L_{d}^{k}) is a value obtained by calculating Expression (21).

In step S27-2, the treatment discriminator optimization unit 127 optimizes the parameters of the treatment discriminator D_{W}. As a result, the parameters of the treatment discriminator D_{W} are updated.

The parameters of the treatment discriminator D_{W} are optimized such that the loss (L_{W}) becomes minimum. An optimization technique such as the well-known stochastic gradient descent is used for the optimization.

The loss (L_{W}) is a value obtained by calculating Expression (31).

In step S28, the encoder learning unit 120 decides whether to repeat the parameter update.

A value obtained by calculating Expression (40) is referred to as a loss (L_{G}^{en}).

If the loss (L_{G}^{en}), the total sum L_{d}, and the loss (L_{W}) are not minimized, the encoder learning unit 120 decides to repeat the parameter update.

If the loss (L_{G}^{en}), the total sum L_{d}, and the loss (L_{W}) are minimized, the encoder learning unit 120 decides not to repeat the parameter update.

If the loss (L_{G}^{en}), the total sum L_{d}, and the loss (L_{W}) are converged, the loss (L_{G}^{en}), the total sum L_{d}, and the loss (L_{W}) have been minimized.

If the loss (L_{G}^{en}), the total sum L_{d}, and the loss (L_{W}) are not minimized but a number of repetition times of processing has reached an upper limit, the encoder learning unit 120 decides not to repeat the parameter update. The upper limit is a predetermined number of times.

If the parameter update is repeated, the processing returns to step S22-1.

If the parameter update is not repeated, the processing proceeds to step S29.

In step S29, the encoder learning unit 120 passes the parameters of each of the generator G^{en}, the treatment dosage discriminator D_{d}, and the treatment discriminator D_{W} to the decoder learning unit 130.

Additionally, the encoder learning unit 120 passes the set Y\hat{ }^{en} in Expression (12) and the set h\hat{ }^{en} in Expression (14) to the decoder learning unit 130.

Returning to FIG. 8, step S30 will be described.

In step S30, the decoder learning unit 130 optimizes a decoder.

The decoder predicts processing results of time t+2 to time t+τ. Time t+2 is time that is 1 step ahead of the time t+1 whose processing result is predicted by the encoder.

Specifically, the decoder learning unit 130 optimizes parameters of the decoder. In other words, the decoder learning unit 130 finds the optimal parameter values for the decoder.

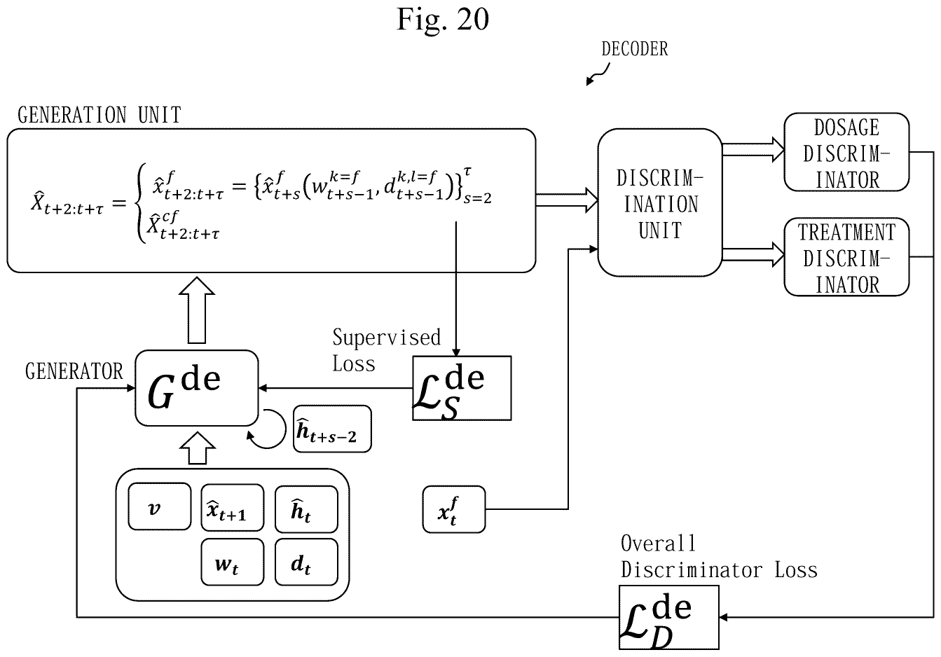

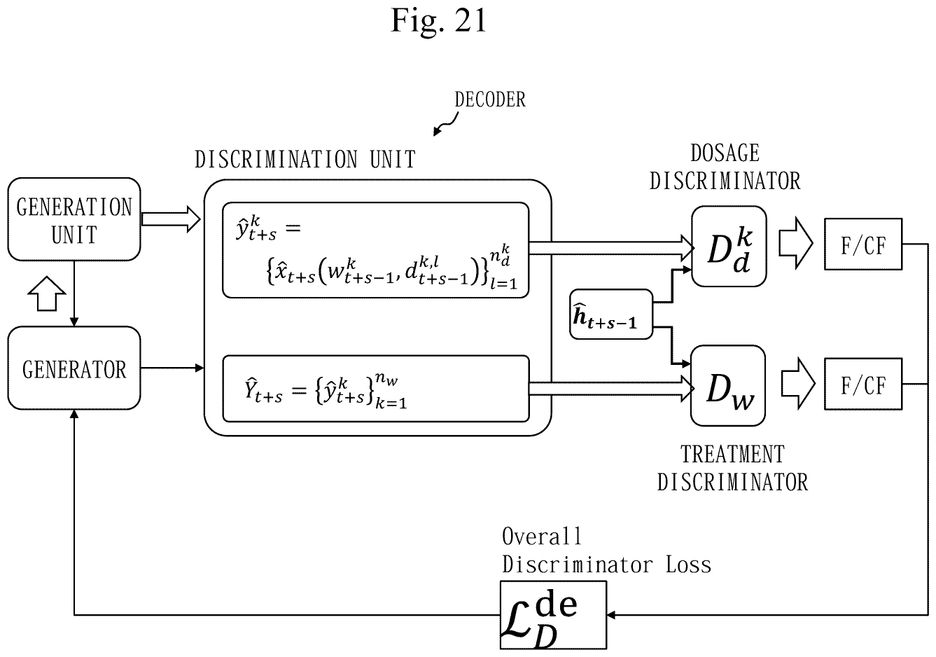

FIG. 20 and FIG. 21 show overviews of a model of the decoder.

The decoder has a generator G^{de}. The decoder furthermore has a treatment dosage discriminator D_{d} and a treatment discriminator D_{W} for each treatment type. The treatment dosage discriminator D_{d} and the treatment discriminator D_{W} are the same as those possessed by the encoder.

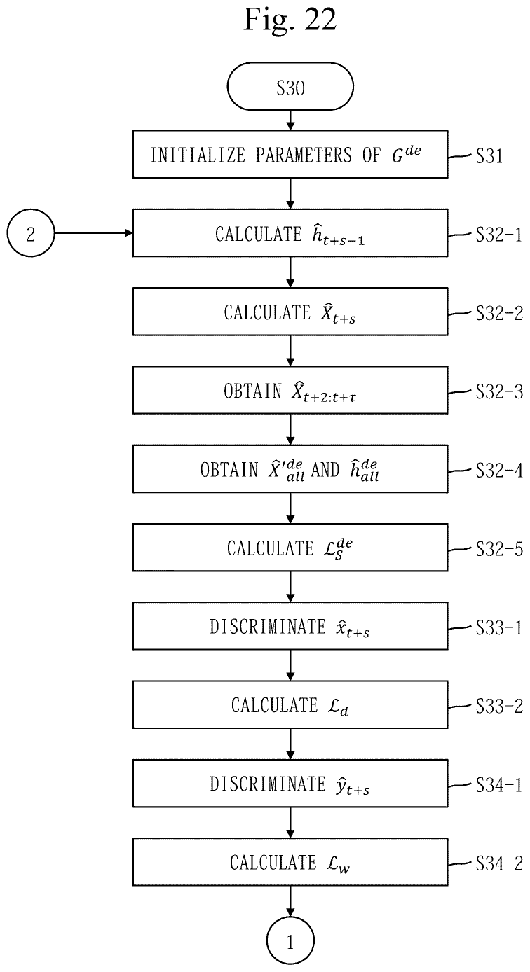

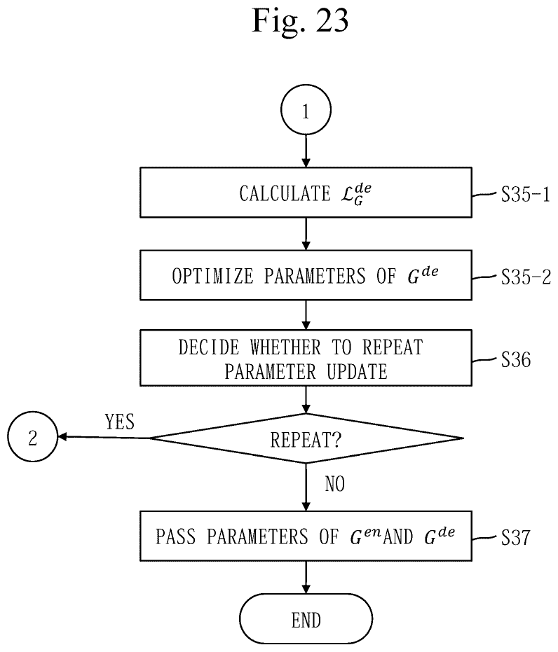

With referring to FIG. 22 and FIG. 23, a procedure of step S30 will be described.

In step S31, the initialization unit 131 initializes parameters of the generator G^{de} using the parameters of the generator G^{en}.

In step S32-1, the generation unit 132 calculates an intermediate state h\hat{ }_{t+S-1} of a recurrent layer of the generator G^{de}.

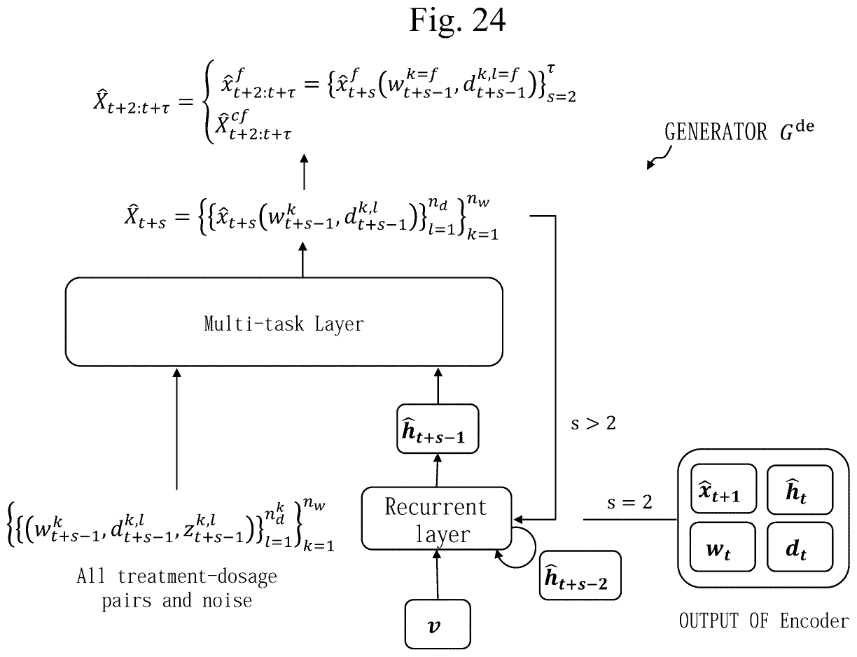

FIG. 24 shows an overview of the generator G^{de}.

In the first step (s=2), the intermediate state h\hat{ }_{t+S-1} is calculated by inputting an element {x\hat{ }ₜ₊₁, wₜ, dₜ}, an element h\hat{ }ₜ, and an element v to the recurrent layer of generator G^{de}.

The element {x\hat{ }ₜ₊₁, wₜ, dₜ} is extracted from the set Y\hat{ }ₜ₊₁ in Expression (10).

The element h\hat{ }ₜ is extracted from the set h\hat{ }^{en} in Expression (14).

Returning to FIG. 22, the explanation resumes from step S32-2.

In step S32-2, the generation unit 132 calculates a set X\hat{ }ₜ₊ₛ.

The set X\hat{ }ₜ₊ₛ is calculated by inputting the intermediate state h\hat{ }_{t+S-1} and a combination of treatment, treatment dosage, and noise to a multi-task layer of the generator G^{de}.

The combination of treatment, treatment dosage, and noise is expressed as follows.

$${\{ {\{ ( {w}_{t + s - 1}^{k} , {d}_{t + s - 1}^{k , l} , {z}_{t + s - 1}^{k , l} ) \}}_{l = 1}^{{n}_{d}^{k}} \}}_{k = 1}^{{n}_{w}} \qquad [ Formula 301 ]$$

The set X\hat{ }ₜ₊ₛ is expressed by Expression (50).

$$\begin{matrix}[ Formula 302 ] & \\ {\hat{X}}_{t + s} = {\{ {\{ {\hat{x}}_{t + s} ( {w}_{t + s - 1}^{k} , {d}_{t + s - 1}^{k , l} ) \}}_{l = 1}^{{n}_{d}} \}}_{k = 1}^{{n}_{w}} & ( 50 )\end{matrix}$$

In step S32-3, the generation unit 132 obtains a set X\hat{ }_{t+2:t+τ}.

The set X\hat{ }_{t+2:t+τ} is obtained by repeating recursive processing from step 2 (s=2) to step t (s=τ).

In the recursive processing, for each element {x\hat{ }ₜ₊ₛ, wₜ₊ₛ₋₁, dₜ₊ₛ₋₁} of the set X\hat{ }ₜ₊ₛ, the intermediate state h\hat{ }ₜ₊ₛ₋₁ and the element v are inputted to the recurrent layer of the generator G^{de} to obtain a set X\hat{ }ₜ₊ₛ₊₁.

The set X\hat{ }_{t+2:t+τ} is expressed by Expression (51).

$$\begin{matrix}[ Formula 303 ] & \\ {\hat{X}}_{t + 2 : t + τ} = \{ \begin{matrix}{\hat{x}}_{t + 2 : t + τ}^{f} = {\{ {\hat{x}}_{t + s}^{f} ( {w}_{t + s - 1}^{k = f} , {d}_{t + s - 1}^{k , l = f} ) \}}_{s = 2}^{τ} \\ {\hat{X}}_{t + 2 : t + τ}^{cf}\end{matrix} & ( 51 )\end{matrix}$$

In the set X\hat{ }_{t+2:t+τ}, the factual data x\hat{ }^{f} is in only one pattern, and all remaining patterns are counterfactual data X\hat{ }^{cf}.

The number of patterns that are the counterfactual data X\hat{ }^{cf} is expressed as follows.

$${( {\sum }_{k = 1}^{{n}_{w}} {n}_{d}^{k} )}^{τ - 1} - 1 \qquad [ Formula 304 ]$$

In step S32-4, the generation unit 132 obtains a set X\hat{ }′^{de} and a set h\hat{ }^{de}.

The set X\hat{ }′^{de} and the set h\hat{ }^{de} are obtained as follows.

First, the generation unit 132 replaces the factual element x\hat{ }^{f} in Expression (51) with the observed value x^{f} to obtain a set X\hat{ }′_{t+2:t+τ}.

The set X\hat{ }′_{t+2:t+τ} is expressed by Expression (52).

$$\begin{matrix}[ Formula 305 ] & \\ {{\hat{X}}^{′}}_{t + 2 : t + τ} = \{ \begin{matrix}{x}_{t + 2 : t + τ}^{f} = {\{ {x}_{t + s}^{f} ( {w}_{t + s - 1}^{k = f} , {d}_{t + s - 1}^{k , l = f} ) \}}_{s = 2}^{τ} \\ {\hat{X}}_{t + 2 : t + τ}^{cf}\end{matrix} & ( 52 )\end{matrix}$$

Then, for each individual i, the generation unit 132 obtains an element {x^{f}, x\hat{ }^{cf}} of the set X\hat{ }′_{t+2:t+τ} to obtain a set X\hat{ }′^{de}.

The set X\hat{ }′^{de} is expressed by Expression (53).

$$\begin{matrix}[ Formula 306 ] & \\ {{\hat{X}}^{′}}_{all}^{de} = {\{ {\{ {{\hat{X}}^{′}}_{t + 2 : t + τ}^{( i )} \}}_{t = 1}^{{t}_{max}^{( i )} - τ} \}}_{i = 1}^{N} & ( 53 )\end{matrix}$$

The set h\hat{ }^{de} is obtained by calculation in the recurrent layer of the generator G^{de}.

The set h\hat{ }^{de} is expressed by Expression (54).

$$\begin{matrix}[ Formula 307 ] & \\ {\hat{h}}_{all}^{de} = {\{ {\{ {\hat{h}}_{t + 1 : t + τ - 1}^{( i )} \}}_{t = 1}^{{t}_{max}^{( i )} - τ} \}}_{i = 1}^{N} & ( 54 )\end{matrix}$$

In step S32-5, the generation unit 132 calculates a loss function L_{S}^{de}.

The loss function L_{S}^{de} calculates a mean squared error (MSE) of the factual element x\hat{ }^{f} in Expression (51) and the observed value x^{f}.

The loss function L_{S}^{de} is expressed by Expression (55).

$$\begin{matrix}[ Formula 308 ] & \\ {ℒ}_{S}^{de} = {\sum }_{i = 1}^{N} {\sum }_{t = 1}^{{t}_{max}^{( i )} - τ} {\sum }_{s = 2}^{τ} {( {x}_{t + s}^{f , ( i )} - {\hat{x}}_{t + s}^{f , ( i )} )}^{2} & ( 55 )\end{matrix}$$

In step S33-1, the treatment dosage discrimination unit 133 discriminates each element x\hat{ }ₜ₊ₛ.

Each element x\hat{ }ₜ₊ₛ is discriminated as follows.

First, the treatment dosage discrimination unit 133 extracts an element y\hat{ }ₜ₊ₛ (1<s<τ) from the set X\hat{ }′^{de} in Expression (53).

The element y\hat{ }ₜ₊ₛ to be extracted is expressed by Expression (60).

$$\begin{matrix}[ Formula 309 ] & \\ {\hat{y}}_{t + s}^{k} = {\{ {\hat{x}}_{t + s} ( {w}_{t + s - 1}^{k} , {d}_{t + s - 1}^{k , l} ) \}}_{l = 1}^{{n}_{d}^{k}} & ( 60 )\end{matrix}$$

Also, the treatment dosage discrimination unit 133 extracts the element h\hat{ }ₜ₊ₛ₋₁ from set h\hat{ }^{de} in Expression (54).

Then, the treatment dosage discrimination unit 133 inputs the element y\hat{ }ₜ₊ₛ and the element h\hat{ }ₜ₊ₛ₋₁ to a treatment dosage discriminator D_{d}^{k} to discriminate each element x\hat{ }ₜ₊ₛ.

In step S33-2, the treatment dosage discrimination unit 133 calculates a total sum L_{d}.

The total sum L_{d} is calculated as follows.

First, for each individual i, the treatment dosage discrimination unit 133 discriminates the element x\hat{ }ₜ₊ₛ that constitutes the element y\hat{ }ₜ₊ₛ of a set indicated below.

$${\{ {\{ {\{ {\hat{y}}_{t + s}^{k} \}}_{s = 2}^{τ} \}}_{t = 1}^{{t}_{max}^{( i )} - τ} \}}_{i = 1}^{N} \qquad [ Formula 310 ]$$

Next, for each treatment k (k=1 . . . nₖ), the treatment dosage discrimination unit 133 calculates a loss function L_{d}^{k} to determine the loss.

The loss function L_{d}^{k} is expressed by Expression (61).

Note that n^{f}ₓ is a number of pieces of factual data of x\hat{ }ₜ₊ₛ in y\hat{ }ₜ₊ₛ.

Note that n^{cf}ₓ is a number of pieces of counterfactual data of x\hat{ }^{k}ₜ₊ₛ in y\hat{ }ₜ₊ₛ.

$$\begin{matrix}[ Formula 311 ] & \\ {ℒ}_{d}^{k} = {\sum }_{i = 1}^{N} {\sum }_{t = 1}^{{t}_{max}^{( i )} - τ} {\sum }_{s = 2}^{τ} [ {\sum }_{{n}_{x}^{f , ( i )}} log {D}_{d}^{k} ( {x}_{t + s}^{f , ( i )} ) + {\sum }_{{n}_{x}^{cf , ( i )}} log ( 1 - {D}_{d}^{k} ( {\hat{x}}_{t + s}^{cf , ( i )} ) ) ] & ( 61 )\end{matrix}$$

Then, the treatment dosage discrimination unit 133 calculates the total sum L_{d} of losses.

The total sum L_{d} is expressed by Expression (62).

$$\begin{matrix}[ Formula 312 ] & \\ {ℒ}_{d} = {\sum }_{k = 1}^{{n}_{w}} {ℒ}_{d}^{k} & ( 62 )\end{matrix}$$

In step S34-1, the treatment discrimination unit 134 discriminates an element y\hat{ }ₜ₊ₛ.

The element y\hat{ }ₜ₊ₛ is discriminated as follows.

First, the treatment discrimination unit 134 extracts an element Y\hat{ }ₜ₊ₛ from the set X\hat{ }′^{de} in Expression (53).

The element Y\hat{ }ₜ₊ₛ is expressed by Expression (63).

$$\begin{matrix}[ Formula 313 ] & \\ {\hat{Y}}_{t + s} = {\{ {\hat{y}}_{t + s}^{k} \}}_{k = 1}^{{n}_{w}} = {\{ {\{ {\hat{x}}_{t + s} ( {w}_{t + s - 1}^{k} , {d}_{t + s - 1}^{k , l} ) \}}_{l = 1}^{{n}_{d}^{k}} \}}_{k = 1}^{{n}_{w}} & ( 63 )\end{matrix}$$

Also, the treatment discrimination unit 134 extracts the element h\hat{ }ₜ₊ₛ₋₁ from the set h\hat{ }^{de} in Expression (54).

Then, the treatment discrimination unit 134 inputs the element Y\hat{ }ₜ₊ₛ and the element h\hat{ }ₜ₊ₛ₋₁ to the treatment discriminator D_{w} to discriminate each element y\hat{ }ₜ₊ₛ.

In step S34-2, for each individual i, the treatment discrimination unit 134 discriminates the element y\hat{ }ₜ₊ₛ that constitutes the element Y\hat{ }ₜ₊ₛ of a set indicated below.

$${\{ {\{ {\{ {\hat{Y}}_{t + s} \}}_{s = 2}^{τ} \}}_{t = 1}^{{t}_{max}^{( i )} - τ} \}}_{i = 1}^{N} \qquad [ Formula 314 ]$$

Then, the treatment discrimination unit 134 calculates a loss function L_{W}.

The loss function L_{W} is expressed by Expression (64).

Note that n^{f}_{y} is a number of pieces of factual data of y\hat{ }ₜ₊ₛ in y\hat{ }ₜ₊ₛ.

Note that n^{cf}_{y} is a number of pieces of counterfactual data of y\hat{ }ₜ₊ₛ in y\hat{ }ₜ₊ₛ.

$$\begin{matrix}[ Formula 315 ] & \\ {ℒ}_{w} = {\sum }_{i = 1}^{N} {\sum }_{t = 1}^{{t}_{max}^{( i )} - 1} {\sum }_{s = 2}^{τ} [ {\sum }_{{n}_{y}^{f , ( i )}} log {D}_{w} ( {\hat{y}}_{t + s}^{k = f , ( i )} ) + {\sum }_{{n}_{y}^{cf , ( i )}} log ( 1 - {D}_{w} ( {\hat{y}}_{t + s}^{k = cf , ( i )} ) ) ] & ( 64 )\end{matrix}$$

In step S35-1, the generator optimization unit 135 calculates a loss function L_{G}^{de} of the generator G^{de} using a loss (L_{S}^{de}), the total sum L_{d}, and a loss (L_{W}).

The loss (L_{S}^{de}) is a value obtained by calculating Expression (55).

The total sum L_{d} is a value obtained by calculating Expression (62).

The loss (L_{W}) is a value obtained by calculating Expression (64).

The loss function L_{G}^{de} is expressed by Expression (70).

$$\begin{matrix}[ Formula 316 ] & \\ {ℒ}_{G}^{de} = {ℒ}_{S}^{de} - {α}_{d} {ℒ}_{d} - {α}_{w} {ℒ}_{w} & ( 70 )\end{matrix}$$

In step S35-2, the generator optimization unit 135 optimizes the parameters of the generator G^{de}. As a result, the parameters of the generator G^{de} are updated.

The parameters of the generator G^{de} are optimized such that an output value of the loss function L_{G}^{de} becomes minimum. An optimization technique such as the known stochastic gradient descent method is used for the optimization.

In step S36, the decoder learning unit 130 decides whether to repeat the parameter update.

A value obtained by calculating Expression (70) is referred to as a loss (L_{G}^{de}).

If the loss (L_{G}^{de}) is not minimized, the decoder learning unit 130 decides to repeat the parameter update.

If the loss (L_{G}^{de}) is minimized, the decoder learning unit 130 decides not to repeat the parameter update.

If the loss (L_{G}^{de}) is converged, the loss (L_{G}^{de}) has been minimized.

If the loss (L_{G}^{de}) is not minimized but a number of times of repetition processing has reached an upper limit, the decoder learning unit 130 decides not to repeat the parameter update. The upper limit is a predetermined number of times.

If the parameter update is repeated, the processing returns to step S32-1.

If the parameter update is not repeated, the processing proceeds to step S37.

In step S37, the decoder learning unit 130 passes the parameters of each of the generator G^{en} and the generator G^{de} to the estimation unit 140.

Returning to FIG. 8, the explanation resumes from step S40.

In step S40, the estimation unit 140 selects a treatment plan to achieve a treatment result.

The treatment result indicates an effect (treatment effect) obtained from a plurality of treatments.

Specifically, the estimation unit 140 estimates a treatment result of each time following time t+1, concerning the plurality of treatments, using the optimized encoder and the optimized decoder. Then, the estimation unit 140 selects a treatment plan that provides a high final treatment result or a treatment plan that provides a high cost performance, based on estimation results concerning the plurality of treatments.

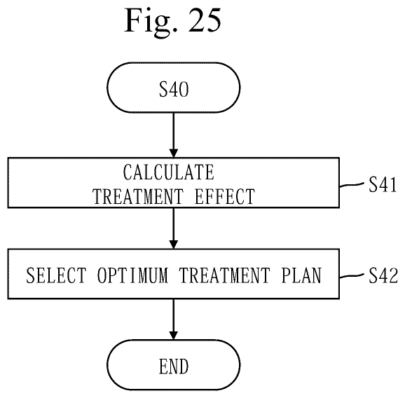

With referring to FIG. 25, the procedure of step S40 will be described.

In step S41, the estimation unit 140 uses the generator G^{en} and the generator G^{de} to calculate a treatment result x\hat{ }_{t+1:t+τ}.

The treatment plan is expressed as follows.

$${\{ {w}_{t + s}^{k} , {d}_{t + s}^{k , l} \}}_{s = 1}^{τ} \qquad [ Formula 401 ]$$

In step S42, the estimation unit 140 selects an optimal treatment plan.

The optimal treatment plan is a treatment plan that provides a high final treatment result x\hat{ }_{t+τ}.

Alternatively, the optimal treatment plan may be a treatment plan that provides a high cost performance in the treatment cost and the treatment result x\hat{ }_{t+τ} where the treatment dosage is regarded as the treatment cost.

The treatment dosage is expressed as follows.

$${\{ {d}_{t + s}^{k , l} \}}_{s = 1}^{τ} \qquad [ Formula 402 ]$$

Returning to FIG. 8, step S50 will be described.

In step S50, the output unit 150 outputs the optimal treatment plan and the treatment result x\hat{ }_{t+1:t+τ} which is obtained by an advantageous treatment plan.

### Effects of Embodiment 1

Embodiment 1 is designed to estimate the treatment results at a plurality of time points where both the treatment and the treatment dosage change as time passes.

The information processing device 100 estimates counterfactual treatment results for a plurality of types of treatments at a plurality of time points and a treatment dosage of each treatment. As a result, when conducting a plurality of times of treatments, the information processing device 100 is able to grasp in advance which treatment and what treatment dosage would provide a good effect in each treatment time, allowing formulation of an accurate treatment plan.

Estimating the treatment results at the plurality of time points in Embodiment 1 enables to grasp in advance which treatment plan should be formulated at what time point according to the characteristic of the target.

For example, it is possible to formulate a treatment plan for a case where vaccine is administered four times (refer to FIG. 1). The treatment plan indicates multiple combinations of treatments, dosage of treatments, a sequence of treatments, and a timing of treatment. Additionally, the treatment plan indicates when to stop treatment, and so on.

### Supplement to Embodiment 1

Embodiment 1 is formed of a first block and a second block.

The first block is a block that “generates a treatment effect from a state and treatment”, and operates as follows. The first block includes comparing a treatment effect generated by a GAN and a treatment effect estimated by the encoder and conducting learning cooperatively. In the first block, three loss functions are calculated.

The second block is a block that “estimates a treatment from a treatment effect”, and operates as follows. The second block includes introducing a GAN or an NN that generates a treatment or classifies a treatment, and estimating a treatment A_{1:t}={W_{1:t}, D_{1:t}} from a treatment effect Yₜ. In the second block, a discriminator is introduced, and whether a pair of estimated treatments {W_{1:t}, D_{1:t}} is factual or counterfactual is decided.

Applications of Embodiment 1 are not limited to a vaccine treatment schedule. Embodiment 1 can be applied to various fields.

For instance, Embodiment 1 can be applied to a distribution plan for sales promotion of products or services. In the distribution plan, items such as discount coupons and advertisements are distributed. Specifically, Embodiment 1 enables formulation of a distribution plan that indicates how many discount coupons and advertisements are to be distributed in what order and at what timing.

For example, Embodiment 1 can be applied to dynamic pricing of train seat reservation or hotel rooms. Specifically, Embodiment 1 enables formation of a pricing plan that indicates how much the pricing for sales promotion of seat reservation or rooms is to be varied in what order and at what timing.

In this manner, Embodiment 1 can be applied to a wide area including supporting medical planning such as a vaccine treatment plan, formulating a distribution plan for sales promotion of products or services, formulating dynamic pricing of train seat reservation or hotel rooms.

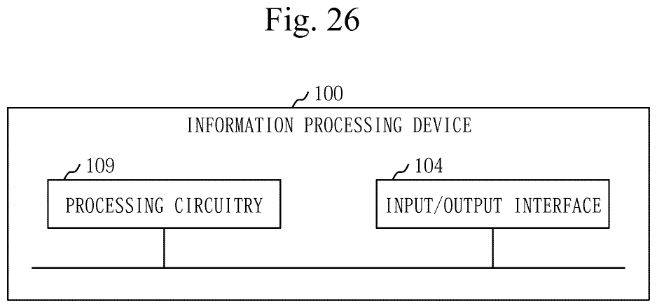

With referring to FIG. 26, a hardware configuration of the information processing device 100 will be described.

The information processing device 100 is equipped with processing circuitry 109.

The processing circuitry 109 is a hardware device that implements the data acquisition unit 110, the encoder learning unit 120, the decoder learning unit 130, the estimation unit 140, and the output unit 150.

The processing circuitry 109 may be a dedicated hardware device, or a processor 101 that executes the program stored in the memory 102.

If the processing circuitry 109 is a dedicated hardware device, the processing circuitry 109 is, for example, a single circuit, a composite circuit, a programmed processor, a parallel-programmed processor, an ASIC, or an FPGA; or a combination of these.

Note that ASIC stands for Application Specific Integrated Circuit.

Note that FPGA stands for Field Programmable Gate Array.

The information processing device 100 may include a plurality of processing circuitries that substitute for the processing circuitry 109.

In the processing circuitry 109, some functions may be implemented by dedicated hardware, while the remaining functions may be implemented by software or firmware.

In this manner, the functions of the information processing device 100 can be implemented by hardware, software, or firmware; or a combination of these.

Embodiment 1 is an exemplification of a preferred embodiment and is not intended to limit the technical scope of the present disclosure. Embodiment 1 may be implemented partially, or may be implemented by combination with other embodiments. The procedures described using the flowcharts and so on may be changed as appropriate.

The term “unit” in the name of each element of the information processing device 100 may be replaced with “process”, “stage”, “circuit”, or “circuitry”.

### REFERENCE SIGNS LIST

100: information processing device; 101: processor; 102: memory; 103: auxiliary storage device; 104: input/output interface; 109: processing circuitry; 110: data acquisition unit; 111: acquisition unit; 112: pre-processing unit; 120: encoder learning unit; 121: initialization unit; 122: generation unit; 123: treatment dosage discrimination unit; 124: treatment discrimination unit; 125: generator optimization unit; 126: treatment dosage discriminator optimization unit; 127: treatment discriminator optimization unit; 130: decoder learning unit; 131: initialization unit; 132: generation unit; 133: treatment dosage discrimination unit; 134: treatment discrimination unit; 135: generator optimization unit; 136: discriminator optimization unit; 140: estimation unit; 150: output unit; 190: storage unit.

1. An information processing device comprising
processing circuitry
to acquire, as training data concerning a plurality of treatments, time-series data including: a variant feature that varies according to treatment; an invariant feature that does not vary according to treatment; and a categorical variable concerning treatment and a treatment dosage,
to optimize an encoder that predicts a treatment result corresponding to treatment and a treatment dosage, of time t+1 that is 1 step ahead of any given time t, using the training data,
to optimize a decoder that predicts a treatment result corresponding to treatment and a treatment dosage, of each time following time t+1, using the training data, and
to estimate a treatment result of each of a plurality of times following time t+1, concerning the plurality of treatments, using the optimized encoder and the optimized decoder,
each of the encoder and the decoder comprising
a generator to generate the treatment result corresponding to treatment and a treatment dosage;
a treatment discriminator to discriminate provided treatment from the treatment result; and
a treatment dosage discriminator to discriminate provided treatment dosage from the treatment result.

2. The information processing device according to claim 1, wherein the processing circuitry selects a treatment plan that provides a high final treatment result or a treatment plan that provides a high cost performance, based on estimation results, and estimates, concerning a plurality of treatments of the selected treatment plan, at least one of a treatment combination, a treatment dosage, and a treatment sequence.

3. An information processing method comprising:
acquiring, as training data concerning a plurality of treatments, time-series data including: a variant feature that varies according to treatment; an invariant feature that does not vary according to treatment; and a categorical variable concerning treatment and a treatment dosage;
optimizing an encoder that predicts a treatment result corresponding to treatment and a treatment dosage, of time t+1 that is 1 step ahead of any given time t, using the training data;
optimizing a decoder that predicts a treatment result corresponding to treatment and a treatment dosage, of each time following time t+1, using the training data; and
estimating a treatment result of each of a plurality of times following time t+1, concerning the plurality of treatments, using the optimized encoder and the optimized decoder,
each of the encoder and the decoder comprising
a generator to generate the treatment result corresponding to treatment and a treatment dosage;
a treatment discriminator to discriminate provided treatment from the treatment result; and
a treatment dosage discriminator to discriminate provided treatment dosage from the treatment result.

4. The information processing method according to claim 3, comprising
optimizing each of the encoder and the decoder by optimizing parameters of each of the generator, the treatment discriminator, and the treatment dosage discriminator using a generative adversarial network.

5. A non-transitory computer readable medium recorded with an information processing program which causes a computer to execute:
a data acquisition process of acquiring, as training data concerning a plurality of treatments, time-series data including: a variant feature that varies according to treatment; an invariant feature that does not vary according to treatment; and a categorical variable concerning treatment and a treatment dosage;
an encoder learning process of optimizing an encoder that predicts a treatment result corresponding to treatment and a treatment dosage, of time t+1 that is 1 step ahead of any given time t, using the training data;
a decoder learning process of optimizing a decoder that predicts a treatment result corresponding to treatment and a treatment dosage, of each time following time t+1, using the training data; and
an estimation process of estimating a treatment result of each of a plurality of times following time t+1, concerning the plurality of treatments, using the optimized encoder and the optimized decoder,
each of the encoder and the decoder comprising
a generator to generate the treatment result corresponding to treatment and a treatment dosage;
a treatment discriminator to discriminate provided treatment from the treatment result; and
a treatment dosage discriminator to discriminate provided treatment dosage from the treatment result.
